(19) United States
(12) Patent Application Publication
(10) Pub. No.: US 2021/0315798 A1
(43) Pub. Date: Oct. 14, 2021

(54) UVB-INDUCED PHOTODAMAGES: COMPOSITIONS AND METHODS FOR TOPICAL TREATMENT

(71) Applicants: Dead Sea and Arava Science Center, D.N. Dead Sea (IL); ICA in Israel, Omer (IL)

(72) Inventors: Zvi BENTWICH, Tel Aviv (IL); Joseph MELOUL, Omer (IL); Dimitry LAN, Be'er-Sheba (IL); Guy COHEN, Kibbutz Ein Gedi (IL)

(21) Appl. No.: 17/265,324

(22) PCT Filed: Aug. 1, 2019
(86) PCT No.: PCT/IL2019/050875

§ 371 Date: Feb. 2, 2021

Related U.S. Application Data
(60) Provisional application No. 62/713,613, filed on Aug. 2, 2018.

(51) Int. Cl. A61K 8/9789; A61Q 19/00; A61Q 17/04

(52) U.S. Cl. CPC A61K 8/9789; A61Q 17/04; A61Q 19/004

(57) ABSTRACT

The present invention is directed to a composition comprising a water-based extract of a Commiphora plant, and method of use thereof for preventing or treating an ultra-violate (UV) radiation damage to a subject's skin.

### CROSS-REFERENCE TO RELATED APPLICATIONS

This application claims the benefit of priority of U.S. Provisional Patent Application No. 62/713,613 titled “COMPOSITIONS AND METHODS FOR TREATING UVB-INDUCED PHOTODAMAGES”, filed Aug. 2, 2018, the contents of which is incorporated herein by reference in its entirety.

### FIELD OF INVENTION

The present invention, in some embodiments thereof, relates to plant extracts and use thereof, such as for preventing or treating damages caused by ultraviolet (UV) radiation.

### BACKGROUND OF THE INVENTION

In recent decades the reported incidence of melanoma and non-melanoma skin cancer has been consistently growing worldwide. These have been primarily ascribed to increased exposure to sun radiation with its strong ultraviolet (UV) radiation. The latter induces direct damage to the cell's DNA and membranes, thus regarded as the major risk factors and causes for skin cancers. UVB-induced carcinogenesis is related to UV absorption by the cell's DNA, that results in damage to the DNA, producing mutagenic dimeric photoproducts, namely cyclobutane-pyrimidine dimers (CPDs) and 6-4 photoproducts (6-4PPs).

Sunscreens which attenuate UV radiation by absorption are the major protective agents against sun exposure. They protect the skin from radiation-induced photobiological changes and usually contain combinations of various active agents which absorb or scatter incident radiation, dispersed in a variety of formulas. The active ingredients are not absorbed by the epidermis, the dermis or into the blood. However, serious concerns regarding their ecotoxicity, photo instability, phototoxicity and photoallergic reactions have been raised and currently remain unresolved. In a study involving 2,715 patients, sunscreen agents (especially benzophenone-3) were the most common causes of photo allergy. This finding, since its initial publication, has been re-confirmed by a second study with over a thousand patients. Controlled human studies have demonstrated that sunscreen usage does not always prevent sunburn. One of the reasons for this dichotomy is that individuals do not apply sunscreens at the same concentrations as utilized in controlled sun protecting factor (SPF) testing—that is, 2 mg/cm′. Other studies have demonstrated that most sunscreen users apply only 25-75% of that quantity. In addition, treatment for reversing sun damages caused by UV radiation overexposure is currently unavailable.

Currently, there is a growing demand in western society for natural and less toxic sun protecting agents. Natural herbal products are gaining more attention as possible and reliable substitutes for synthetic sunscreens, especially those shown to have antioxidant, UV absorbance, and/or anti-inflammatory properties. Safe and efficient compositions, particularly cosmetic and skin care compositions that are useful in treating or preventing the damage of sun radiation and in keeping skin cells viability, while not including potentially harmful synthetic compounds, are still being pursued. To this end, such protection is desired not only for healthy individuals but also for patients with high sensitivity to light (photodermatitis), for which even short term exposure to sunlight may lead to harmful impact, ranging from skin erythema to cancer formation.

### SUMMARY OF THE INVENTION

In some embodiments, the present invention is directed to a composition comprising a water-based extract of a Commiphora plant. In some embodiments, the present invention is directed to a composition comprising an extract obtained by water-based extraction of a Commiphora plant. In some embodiments, the present invention is directed to methods for preventing or treating an ultra-violate (UV) radiation damage to a subject's skin using the compositions of the invention. In some embodiments, the invention is based, in part, on the findings that C. gileadensis water-based extracts are highly effective as UV radiation protectants.

According to one aspect, there is provided a composition comprising a water-based extract of a Commiphora plant.

According to another aspect, there is provided a composition comprising an extract obtained by water-based extraction of Commiphora plant.

In some embodiments, the composition comprises: (a) leaves: 10-30% (w/w) by dry weight; (b) fruits: 5-15% (w/w) by dry weight; (c) branches: 20-60% (w/w) by dry weight; or any combination thereof. In some embodiments, the composition comprises at least 10% (w/w) sap by dry weight.

In some embodiments, the composition comprises 0.2 to 2% (w/w) of a phenolic compound. In some embodiments, the composition comprises 0.01 to 0.05% (w/w) of a volatile organic compound (VOC).

In some embodiments, Commiphora is Commiphora gileadensis.

In some embodiments, the composition comprises a fraction of a water-based extract.

In some embodiments, the composition further comprises an anti-oxidant, a chelator, a cleansing agent, a skin protectant, a sunscreen, a skin lightening agent, an anti-wrinkling agent, an anti-inflammatory agent, an anti-aging agent, or any combination thereof.

In some embodiments, the composition comprises a dermatologically acceptable carrier. In some embodiments, the composition comprises a cosmetically acceptable carrier. In some embodiments, the composition is in the form of a cream, a lotion, an ointment or a spray.

In some embodiments, the composition is topically applied to the subject's skin before, during, or after exposure to UV radiation. In some embodiments, UV radiation is UVB radiation. In some embodiments, the subject is at risk of sunlight exposure.

According to another aspect, there is provided a method for preventing or treating an ultra-violate (UV) radiation damage to a subject's skin, comprising topically applying to the subject's skin a composition comprising an effective amount of a water-based extract of Commiphora plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B are chromatograms of gas chromatography analyses of WSE (1A) and WPE (1B) of C. gileadensis.

FIG. 2 is a vertical bar graph showing phenolic contents (gray bars) and antioxidant capacity (black bars) of whole sap emulsion (WSE) and whole plant emulsion/extract (WPE) of C. gileadensis.

FIGS. 3A-3E are vertical bar graphs and an illustration demonstrating that WPE and WSE of C. gileadensis attenuate UVB induced apoptosis in human skin explants organ culture (HSOCs). (3A) is a vertical bar graph showing the dynamic C. gileadensis's photoprotective activity throughout July. HSOCs were pretreated topically with WSE (0.8 mg/cm²) and WPE (0.9 mg/cm²) for 24 hours. Following incubation, the explants were challenged with UVB (400 mJ/cm2). Caspase-3 activity levels were measured 24 hours post-irradiation. (3B) is a vertical bar graph showing the dynamic C. gileadensis's cytotoxicity throughout July. HSOCs were pretreated topically with WSE and WPE for 24 hours. Then, the skin was exposed to UVB. Viability (MTT assay) was measured 24 hours post-irradiation. Values are mean±SEM; n=4. (3C) is a non-limiting schematic representation of the change in the effect caused by C. gileadensis WPE during the period from July to August. It seems that there is a positive conversion in the activity of C. gileadensis WPE occurring between the first and second half of July. (3D) is a vertical bar graph showing that the photoprotective effect of WPE with respect to apoptosis reduction in HSOCs is dose dependent. (3E) is a vertical bar graph showing the differential activity components of WPE. The stem of the plant was taken, the hard bark was separated from the pith, and apoptosis of HSOCs was evaluated as mentioned above. It was showed that the main reservoir of the substance responsible for the photoprotective activity and cytotoxicity, is in the bark of the plant, and not in its pith.

FIGS. 4A-4B are vertical bar graphs showing C. gileadensis retains its photoprotective ability under multiple irradiations. WSE and WPE of C. gileadensis retained their photoprotective activity upon repeated exposure to harmful sunlight on HSOCs. The HSOCs were pretreated topically with WSE (0.8 mg/cm²), WPE (0.9 mg/cm²) and mixture of WSE/WPE 1:1 (v/v) for 24 hours. Then, HSOCs were exposed to UVB. The next day, the HSOCs were irradiated again. Apoptosis (4A) and viability (4B) were measured 24 hours post-irradiation. Values are mean±SEM; n=4. * indicates P<0.05 with respect to non-pretreated control; # indicates P<0.05 with respect to UVB irradiated control.

FIGS. 5A-5B are vertical bar graphs showing C. gileadensis obtained in Ein-Gedi, Israel (7 Aug. 2016), proved to be advantageous over other available ethereal Myrrha oils in a comparative analysis. The WPE and WSE of local C. gileadensis were shown to be more effective than ethereal Myrrha oils from other regions of the Earth. HSOCs were pretreated topically with WSE, WPE and other commercial products for 24 hours. Left side of the graph: controls, WSE and WPE; in the right side of the graph: commercial, Ethiopian and Yemeni ethereal oils. Then, HSOCs were exposed to UVB. Apoptosis (5A) and viability (5B) were measured 24 hours post-irradiation. Values are mean±SEM; n=4. * indicates P<0.05 with respect to non-pretreated control; # indicates P<0.05 with respect to UVB irradiated control.

FIGS. 6A-6D are vertical bar graphs showing C. gileadensis obtained in Ein-Gedi, Israel (7 Aug. 2016), proved to be advantageous in comparison with commercial sunscreens. The HSOCs were pretreated topically with creams containing UV-blocker ZnO (zinc oxide), WSE or WPE for 24 hours. Left side of the graph: controls and cream-ZnO; in middle: cream-WSE; right side: cream-WPE. Then, HSOCs were exposed to UVB. Apoptosis (6A) and viability (6B) were measured 24 hours post-irradiation. The local WPE and WSE of C. gileadensis were less cytotoxic for HSOCs than the most popular commercial sunscreen agents. The HSOCs were pretreated with WPE and mineral sunscreens with various sun protection factor (SPF; 15-100) for 24 hours. Left side of the graph: controls and cream-WPE; right side: sunscreen products. Then, HSOCs were exposed to UVB. Apoptosis (6C) and viability (6D) was measured 24 hours post-UVB. Values are mean±SEM; n=4. * indicates P<0.05 with respect to non-pretreated control; # indicates P<0.05 with respect to UVB irradiated control.

FIGS. 7A-7C are vertical bar graphs showing that adding WPE and WSE of local C. gileadensis to sunscreen lotion causes a powerful synergistic effect of photoprotection. HSOCs pretreated with C. gileadensis WPE and WSE and sunscreen lotion, showed a lower level of UVB-induced apoptosis. The C. gileadensis emulsion in various concentrations was mixed with suntan lotion (30 SPF). HSOCs were pretreated topically with manufactured suntan lotion-emulsion for 24 hours. Left side of the graph: controls, WPE and original sunscreen lotion spray (LS); in middle: mixtures of 25% LS and WPE; right side: mixtures of 10% LS and WPE. Then, HSOCs were exposed to UVB. Apoptosis (7A) and viability (7B) were measured 24 hours post-irradiation. Values are mean±SEM; n=4. * indicates P<0.05 with respect to non-pretreated control; # indicates P<0.05 with respect to UVB irradiated control. (7C) HSOCs pretreated with C. gileadensis WPE and WSE and sunscreen lotion, demonstrated a lower level of UVB-induced photolesions. Analysis of the formation of CPDs in epidermal cells subjected to pretreatment with freshly prepared emulsions of C. gileadensis (16 Aug. 2016) and subsequent exposure to UVB radiation. Dimer formation was measured by ELISA. HSOCs were pretreated topically with manufactured lotion-emulsion for 24 hours. Left side of the graph: controls, emulsions (WSE and WPE); right side: original sunscreen lotions spray (LS) and mixtures of LS and 10% emulsions. Then, HSOCs were exposed to UVB. DNA photolesions were tested following post-irradiation. Values are mean±SEM; n=4. * indicates P<0.05 with respect to non-pretreated control; # indicates P<0.05 with respect to UVB irradiated control.

FIGS. 8A-8D demonstrate C. gileadensis WPE and WSE have a dual anti-inflammatory effect. (8A-8C) are vertical bar graphs showing C. gileadensis WPE and WSE down-regulated pro-inflammatory cytokine production in HSOCs. After topical application of C. gileadensis WPE and WSE to HSOC inflammatory model, i.e., HSOCs that have been previously exposed to LPS (lipopolysaccharides) and EGF (epidermal growth factor) and after forty-eight hours incubation, the medium was collected to perform ELISA test for determining the amount of pro-inflammatory cytokines IL-6 (8A), IL-8 (8B) and TNFα (8C) in the medium of the HSOCs. Values are mean±SEM; n=4. * indicates P<0.05 with respect to untreated control; # indicates P<0.05 with respect to the LPS plus EGF-induced control. (8D) is images of western blot analysis of heme oxygenase-I (HO-1) and the corresponding vertical bar graph of densitometry showing that C. gileadensis WPE and WSE increased the expression of the HO-1 enzyme. Western blot analysis of HO-1 expression in HSOCs after pro-inflammatory stimulation with EGF plus LPS was performed using primary anti-HO-1 antibody; anti-β-actin was used as a loading control. Bar graph shows densitometry analysis of HO-1 signals after normalization with β-actin. Image analysis was done by the ImageJ software. Evaluation of HO-1 levels in HSOCs following induction of inflammation by EGF and LPS and addition of WPE or WSE cultured for 48 hours and then analyzed. Values are mean±SEM; n=3. * indicates P<0.05 with respect to non-pretreated control; # indicates P<0.05 with respect to EGF+LPS-induced control.

### DETAILED DESCRIPTION OF THE INVENTION

In some embodiments, the present invention is directed to a composition comprising a water-based extract of a Commiphora plant. In some embodiments, the present invention is directed to a composition comprising an extract obtained by water-based extraction of a Commiphora plant. In some embodiments, the present invention is directed to methods for preventing or treating an ultra-violate (UV) radiation damage to a subject's skin using the compositions of the invention.

### Extracts and Compositions

In some embodiments, the present invention is directed to plant extracts. In some embodiments, a plant extract of the invention is derived from a member of the Commiphora genus (also known as “Myrrh”). In some embodiments, a member of the Commiphora genus is C. gileadensis.

As used herein, an extract of the present invention comprises the whole extract, a fraction thereof, a portion thereof, an isolated compound therefrom, or any combination thereof.

In some embodiments, the C. gileadensis plant extracts are solvent-based extracts obtained from any selected part of Commiphora gileadensis by a solvent extraction method. In some embodiments, the solvent according to the invention is a polar solvent. In some embodiments, a polar solvent is water or any aqueous solution, such as water-based buffers or media. Non-limiting examples of water-based buffer include, but are not limited to Phosphate Buffered Saline (PBS), Tris, 4-(2-hydroxyethyl)-1-piperazineethanesulfonic acid (HEPES), 3-(N-morpholino)propanesulfonic acid (MOPS), all of which would be apparent to one of ordinary skill in the art. In some embodiments, the polar solvent is compatible with mammalian skin.

In some embodiments, the plant material used in the extraction process comprises the entire plant. In some embodiments, the plant material used in the extraction process comprises one or more distinct tissues from the plant. In some embodiments, the plant material used in the extraction process comprises leaves, stems, fruits, roots, sap, or any combination thereof.

In one embodiment, the composition comprises plant material comprising leaves. In one embodiment, the quantity of leaves in the plant material by dry weight is 5-20% (w/w), 10-35% (w/w), 15-40% (w/w), 20-45% (w/w), 30-55% (w/w), 40-60% (w/w), 50-70% (w/w), or 45-75% (w/w) of the composition by dry weight. Each possibility represents a separate embodiment of the present invention. In one embodiment, the composition comprises plant material comprising fruits. In one embodiment, the quantity of fruits in the plant material by dry weight is 1-10% (w/w), 5-15% (w/w), 8-20% (w/w), 12-25% (w/w), 10-35% (w/w), 17-30% (w/w), or 20-40% (w/w). Each possibility represents a separate embodiment of the present invention. In one embodiment, the composition comprises plant material comprising branches. In one embodiment, the quantity of branches in the plant material by dry weight is 1-5% (w/w), 4-10% (w/w), 8-18% (w/w), 10-30% (w/w), 15-25% (w/w), 20-28% (w/w), or 25-35% (w/w). Each possibility represents a separate embodiment of the present invention. In one embodiment, the composition comprises plant material comprising sap. In one embodiment, the quantity of sap in the plant material by dry weight is 1-10% (w/w), 5-20% (w/w), 8-25% (w/w), 20-40% (w/w), 30-55% (w/w), 50-70% (w/w), 65-90% (w/w), or 85-99% (w/w). Each possibility represents a separate embodiment of the present invention.

As defined herein, the term “sap” refers to a fluid transported in xylem tubes or phloem cells of a plant. In some embodiments, the sap is collected from fresh buds. In some embodiments, the sap collected from fresh bud is centrifuged and the supernatant is collected and dissolved in a water-based solvent. In some embodiment the sap is collected from dried resin. In some embodiments, the dried resin is collected and washed. In some embodiments, the dried resin is ground to powder. In some embodiments, the sap is extracted from a resin ground powder dissolved in a water-based solvent.

In one embodiment, “water-based” is a composition comprising at least 80% w/w water. In one embodiment, “water-based” is a composition comprising at least 85% w/w water. In one embodiment, “water-based” is a composition comprising at least 90% w/w water. In one embodiment, “water-based” is a composition comprising at least 95% w/w water. In one embodiment, “water-based” is a composition comprising at least 97% w/w water. In one embodiment, “water-based” is an aqueous composition.

In some embodiments, the plant material is extracted immediately after harvest. In some embodiments, the plant material is first dried and then extracted. In some embodiments, the plant material is first stored and then extracted. In some embodiments, the raw plant material is first treated and then stored. As used herein, treatment before storage comprises, for example, freezing, drying, lyophilizing, or any combination thereof. In some embodiments, storage period is days to weeks, weeks to months, months to years, or any range therebetween. In some embodiments, the plant material is kept away from light before storage, after storage, or both.

In some embodiments, the plant material is further processed prior to the extraction procedure in order to facilitate the extraction procedure. In some embodiments, processing methods prior to extraction, include but are not limited to crushing, slicing, or shredding, such as by using a grinder or other devices to fragment the plant parts into small pieces or powder. In some embodiments, a processing method prior to extraction includes, but is not limited to washing, such as by water, a buffer, a preservative-containing buffer, or an acidic solution.

In some embodiments, the volume or amount of the solvent to be used in the extraction procedure is proportional to that of the solid plant material. In one embodiment, the proportion is weight/weight (w/w). In some embodiments, the amount of solvent used in the extraction procedure ranges from ×1 to ×100 (mass/mass) that of the solid plant material. In some embodiments, a range of ×1 to ×100 (mass/mass) is ×1 to ×10 (mass/mass), ×5 to ×50 (mass/mass), ×2 to ×40 (mass/mass), ×3 to ×80 (mass/mass), ×7 to ×25 (mass/mass), ×1 to ×20 (mass/mass), ×10 to ×90 (mass/mass), ×15 to ×85 (mass/mass), ×4 to ×30 (mass/mass), ×25 to ×60 (mass/mass), ×40 to ×90 (mass/mass), ×60 to ×85 (mass/mass), ×80 to ×100 (mass/mass), or any range therebetween. Each possibility represents a separate embodiment of the present invention.

In some embodiments, the extraction procedure comprises incubating the plant material over a period ranging between 5 minutes to 24 hours. In some embodiments, ranging between 5 minutes to 24 hours comprises 5 minutes to 30 minutes, 20 minutes to 60 minutes, 1 hours to 3 hours, 2 hours to 5 hours, 4 hours to 10 hours, 8 hours to 16 hours, 12 hours to 18 hours, 15 hours to 20 hours, 19 hours to 24 hours, or any range therebetween. In some embodiments, the extraction procedure comprises incubating the plant material at a temperature ranging between 4° C. to 90° C. In some embodiments, ranging between 4° C. to 90° C. comprises 4° C. to 30° C., 5° C. to 40° C., 10° C. to 70° C., 30° C. to 65° C., 50° C. to 85° C., 55° C. to 75° C., 60° C. to 80° C., 40° C. to 70° C., 10° C. to 50° C., or any range therebetween. Each possibility represents a separate embodiment of the present invention.

In some embodiments, following the extraction procedure, the extracted liquid fraction is separated from the solid (insoluble) matter. Separation of the liquid and solid fractions are achieved by one or more standard separation processes known to one of ordinary skill in the art, and include, but are not limited to various centrifugation processes, filtration processes, or any combination thereof. In some embodiments, the extracted liquid fraction is further subjected to one or more steps of purification. Non-limiting examples of purification methods include, but are not limited to, solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilization, evaporation, precipitation with various “carriers” (including polyvinylpolypyrrolidone (PVPP), carbon, antibodies, and the like), the use of supercritical fluids (such as CO₂), or combinations thereof.

In some embodiments, the extract of the invention is formulated into a composition for topical administration. In some embodiments, the extract is formulated in a form selected from: gel, foam, cream, ointment, emulsion, lotion, powder, suspension or a spray. Each possibility represents a separate embodiment of the invention.

In some embodiments, the composition of the invention further comprises an acceptable cosmetic agent, such as known in the art. In some embodiments, the cosmetic agent is selected from: xanthines, retinoids, a-hydroxy acids, β-hydroxy acids, a-2 adrenergic inhibitors, β-adrenergic agonists, aromatase inhibitors, anti-estrogens, hydroquinone, ascorbic acid, kojic acid, corticosteroids, mucopolysaccharides, collagen, estrogens, isoflavonoids, cinnamic acid, benzoyl peroxide, tropolone, catechol, mercaptoamine, niacinamide, tocopherol, ferulic acid, azelaic acid, botulinum, urea, a derivative, salt thereof, or any combination thereof. Each possibility represents a separate embodiment of the invention.

In some embodiments, the composition further comprises a cosmetically acceptable diluent, carrier or excipient. In one embodiment, the carrier comprises a liposome. In one embodiment, the carrier comprises a micelle. In one embodiment, the carrier comprises a microcapsule. In one embodiment, the carrier comprises any combination of a liposome, a micelle or a microcapsule.

In some embodiments, the composition further comprises an acceptable additive conventionally used in cosmetic and dermatological preparations as is known to a person skilled in the art.

In some embodiments, the composition further comprises an anti-oxidant, a chelator, a cleansing agent, a skin protectant, a sunscreen, a skin lightening agent, an anti-wrinkling agent, an anti-inflammatory agent, an anti-aging agent, or any combination thereof.

As used herein, “an anti-inflammatory agent” refers to any component which facilitate inhibition or suppression of inflammation on or in the skin or in adjacent bodily tissues and thereby reduce redness and swelling of the skin. Non-limiting examples of anti-inflammatory components include, but are not limited to, vitamin E or derivatives thereof, zinc, allantoin, glycyrrhetic acid, azulene, mefenamic acid, phenylbutazone, indomethacin, ibuprofen, ketoprofen, epsilon-aminocaproic acid, hydrocortisone, panthenol or derivatives or salts thereof, zinc oxide and diclofenac sodium.

In some embodiments, the composition further comprises one or more anti-oxidants. In some embodiments, an anti-oxidant comprises enzymatic or non-enzymatic anti-oxidant. Non-limiting examples of enzymatic anti-oxidants include, but are not limited to, superoxide dismutase (SOD), catalase, and glutathione peroxidase. Non-limiting examples of non-enzymatic anti-oxidants include, but are not limited to, Vitamin E (Tocopherol) or derivatives thereof, Vitamin A (Retinol), Vitamin C (Ascorbic acid), carotenoids or derivatives thereof, beta-carotene, canthaxanthin, zeaxanthin, lycopen, lutein, crocetin, capsanthin echinacoside, caffeoyl derivatives, oligomeric proanthocyanidins or proanthanols, green tea polyphenols, dibutyl hydro xytoluene, butyl hydroxyanisole, tannin or derivatives thereof, gallic acid, ellagic acid, flavonoids, flavone, catechin, quercetin, leucoanthocyanidin, quinones, ubiquinone, Vitamin K, thiamines or salts thereof, riboflavin and riboflavin acetate, pyridoxines, pyridoxine hydrochloride, pyridoxine dioctanoate, nicotinic acid, nicotinic acid anmide, benzyl nicotinate, bihirubin, mannitol, tryptophane, histidine and nordihydroguaiaretic acid.

In some embodiments, the composition further comprises vitamin C in the form of ascorbyl palmitate, dipalmitate L-ascorbate, sodium L-ascorbate-2-sulphate, or an ascorbic salt, such as sodium, potassium, or calcium, or mixtures thereof. In some embodiments, the composition further comprises vitamin C in an amount ranging from 0.1 to 50% (w/w). In some embodiments, the composition further comprises vitamin A in the form of vitamin A palmitate. In some embodiments, the composition further comprises vitamin A in an amount ranging from 0.5 to 15% (w/w). In some embodiments, the composition further comprises one or more carotenoids, derivatives thereof or any mixture thereof in an amount ranging from 0.1 to 5% (w/w).

In some embodiments, the composition of the present invention further comprises a compound having sunscreen and/or sunblock properties. Non-limiting examples include, but are not limited to, titanium dioxide, zinc oxide, talc, red veterinary petrolatum, a cinnamate (such as octyl methoxycinnamate), a benzone (such as oxybenzone or 2-hydroxy-4-methoxy benzophenone), a salicylate (such as homosalicylate or octyl salicylate), a benzoic acid (such as para-aminobenzoic acid), and a benzophenone (such as oxybenzophenone). The exact amount of sunscreen employed in the composition will vary depending upon the degree of protection desired from the sun's UV radiation and can be readily determined by one of ordinary skill in the art.

In some embodiments, the present invention is directed to a composition comprising an effective amount of a water-based extract of a Commiphora plant for use in treating and/or preventing a damage caused by UV radiation to a subject's skin cells or skin tissue.

Non-limiting examples of a skin damage include, but are not limited to formation of a wound on the skin, imbalanced skin microbiome, redness, wrinkles, lesions, and others.

In some embodiments, the present invention is directed to a composition for use in reducing inflammation of a UVR-damaged skin. In some embodiments, the composition is for use in wound healing. In some embodiments, the composition is for use in dermatological or cosmetical procedures. In some embodiments, the composition is for use in anti-aging procedures. In some embodiments, the composition is for use in skin microbiome balancing.

The term “dermatological”, as used herein with respect to methods, procedures and compositions of use therein, encompasses therapeutics or cosmetics, or both.

### Methods of Treatment or Prevention

In some embodiments, the present invention is directed to a method for preventing or treating an ultra-violate (UV) radiation damage to a subject's skin, comprising topically applying to the subject's skin a composition comprising an effective amount of a water-based extract of Commiphora plant.

As used herein, “UV radiation damage” refers to any harm to a cell of any of the skin layers as the result of exposure to ultraviolet radiation as described herein. In some embodiments, harm to a cell comprises damages to the cell's: DNA, RNA, proteins, membranes, metabolites, or any combination thereof. In some embodiments, harm to a cell results in apoptosis, necrosis or any type of cell death. In some embodiments, harm to a cell comprises cancerous transformation of the cell. As defined herein, “cancerous transformation” refers to the onset of cancer in the harmed cell. As used herein, “cancer” encompasses diseases associated with cell proliferation.

In some embodiments, UV radiation damage results in skin disease. In some embodiments, a skin disease comprises cancer. In some embodiments, cancer comprises melanoma. In some embodiments, the disease comprises photodermatoses. In some embodiments, the disease comprises Actinic keratoses (AK).

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described composition prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

The term “subject” as used herein refers to an animal, more particularly to non-human mammals and human organism. Non-limiting examples of non-human animals include: horse, cow, camel, goat, sheep, dog, cat, non-human primate, mouse, rat, rabbit, hamster, guinea pig, pig. In one embodiment, the subject is a human. In some embodiments, a subject in need thereof is a subject afflicted with and/or at risk of being afflicted with a condition associated with a skin disease. In one embodiment, a skin disease is induced by exposure to UV radiation.

As used herein, the term “condition” includes anatomic and physiological deviations from the normal that constitute an impairment of the normal state of the living animal or one of its parts, that interrupts or modifies the performance of the bodily functions.

Any concentration ranges, percentage range, or ratio range recited herein are to be understood to include concentrations, percentages or ratios of any integer within that range and fractions thereof, such as one-tenth and one-hundredth of an integer, unless otherwise indicated.

Any number range recited herein relating to any physical feature, such as weight, is to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Materials and Methods

Whole Plant Emulsion/Extract (WPE)—The “whole plant extract” is an emulsion which contains the whole bark and core of the plant and is created when the oleo-gum-resin and all the other parts of the plant are mixed with water and heated. The WPE was obtained using either fresh plants (wet) in a wet process or using dried plants in a dry process.

Dry process—All parts of the plant(branches, leaves, fruits) were cut upside down in a dark, warm and ventilated space, followed by grinding and pulverizing of the resulting dried plant to obtain a powder. Next, the dried plant materials, in proportions of about 50% leaves, 30% fruits and 20% branches were put together and washed with water and 1% (v/v) acetic acid. The materials were at room temperature for 15 minutes, and thereafter were put in a dark and ventilated space and cut into manageable pieces. The dried plant materials were then grinded to obtain powder. Water was then added to the powder as follows: 3 volumes of wet plant material=1 volume of dry material, and from 1 kg of wet plant one can obtain 1.2 kg of WPE. For 50 gr of dry plant powder, 160 ml of water are added; and then incubated at 60° C. for an hour (with frequent stirring) and then cooled. After cooling the liquid is filtered, and then centrifuged at 1,500 RPM for 15 minutes. The resulting sedimented material is then subjected to a press to expel the oils, gum and extract, and then resuspended in 160 ml of water.
Wet process—First, the plant was washed, and cut into manageable pieces, and then a quantity of water corresponding to the potency of the extract required was added. Next, a pulverizer (a juicer) was used to obtain a grinded mixture with a fine texture. This semi-liquid mixture was then incubated at 60° C. for an hour, and then left to cool. After cooling the mixture is subjected to a press to expel the oils, gum and extract, and then filtered. As the mixture is sensitive to light, it has to be kept in a dark container.
WSE—Whole Sap Emulsion (Extract)—Immediately after wounding a Commiphora plant, a clear concentrated liquid seeps out of the wound (Pre-sap or CgSE), which then becomes cloudy and thicker (latex-like thick liquid). If it is left to stand on the tree (or left to drip to the floor) it turns to hard “creamy” resin. The hardened resin (Sap) is then washed, cleaned, and grinded into a 100 micron powder, and filtered to obtain a powder. Water, without or with a preservative agent is added to the powder so that from the solution of 1 kg of Sap, 3-5 liters of emulsion is obtained. The water weight was calculated as (Sap weight×3)×1.2. The emulsion is incubated at 60° C., for 1 hour (stirred frequently), and then centrifuged at 1,500 RPM for 15 minutes. The supernatant is then filtered, and the resultant WSE sealed and melted with Beeswax, transferred to a container covered with aluminum foil, and stored in −80° C. protected from light.

### Cream Preparations

Several creams containing active ingredients can be prepared and evaluated for their effectiveness in skin photoprotection, both before and after exposure to UVB, as described herein. Zinc Oxide (ZnO) cream−75% DDW+14% ZnO+7% Sweet almond oil+4% Sepigel-305. WPE cream−73.14% DDW+17.31% WPE+5.7% Sweet almond oil+3.85% Sepigel-305. WSE cream−66.13% DDW+16.67% WSE+12.5% Sweet almond oil+4.7% Sepigel-305.

### Human Ex-Vivo Skin Explants in Organ Culture (HSOCs)

Full human skin explants in organ culture (HSOCs) have been used as experimental models for intact human skin. The human skin was obtained from adult healthy donors (female; 20-60 y/o), undergoing abdominal reduction surgery. All experiments were conducted with approval of the Institutional Review Board (Ethics “Helsinki” Committee) of Soroka Medical Center, Be′er-Sheva, Israel. The skin explants were used no longer than 12 hours after surgery. Briefly, the samples were cut into pieces of 0.8×0.8 cm² using a mechanical section press (designated press apparatus). The explants were placed in sterile 6-well plates; epidermal side exposed to air, in high glucose DMEM media (Life Technologies Ltd., Gibco®, with 4,500 mg/L D-Glucose+L-Glutamine, w/o Sodium Pyruvate, Cat. No.: 41965-039) supplemented with 1% antibiotics (Penicillin-Streptomycin, Amphotericin B Solution; Biological Industries Israel Beit-HaEmek Ltd., Cat. No.: 03-033-1B). The explants were used after an overnight recovery at 37° C. and 5% CO₂.

### Ultraviolet-B (UVB) Radiation Challenge of HSOCs

Each experiment consisted of quadruplicates for each treatment. WPE, WSE, or a combination of both were applied topically on the epidermis (0.9 or 0.8 mg/cm² outer surface of skin piece, respectively); Additionally, to determine the potency of WPE, a dose-response analysis was performed with decreasing WPE concentrations. WPE was diluted in water and applied in various concentrations on HSOCs. Following incubation for 24 hours, the growth medium was aspirated and replaced with phosphate-buffered saline (PBS×1). The skin pieces (HSOCs) were exposed to UVB irradiation challenge in a dose-response manner (400-750 mJ/cm²). Subsequently, the PBS×1 was aspirated and replaced with fresh medium. HSOCs were then allowed to recover at standard conditions for 24 hours. For collection of the epidermis, the skin was incubated for 1 min in PBS×1 at 56° C., after which the epidermis was physically detached from the dermis using forceps and scalpel and washed again in PBS×1. The epidermis was tested using the techniques described hereinbelow.

Time-course analysis was conducted in the following manner: the HSOCs were cut and pretreated that day and put in incubation; after 24 hours were taken for irradiation and returned for incubation up to collective harvesting day; (non-) pretreated controls were common to all irradiated groups.

### Apoptosis Measurements by Caspase-3 Activity Assay

UVB induced apoptosis in epidermal cells was determined using capase-3 enzymatic activity fluorescence assay. The epidermis pieces were placed in a 96-well plate, and 125 μL of caspase-3 specific substrate solution (500 mM DTT, 10% Triton X-100, caspase-3 substrate diluted 1:1,000 in PBS×1) were added to each well. The enzyme's fluorescent product was measured 20 times at 2-min intervals using a microplate reader, according to the supplier's instructions (37° C.; 40 min; Ex. 355 nm; Em. 460 nm, Calbiochem, Ltd., Cat. No.: 235425).

To rule out the possibility that WPE, WSE, pith extract or bark extract interact directly and inhibit the proteolytic activity of caspase-3, human recombinant C-terminal histidine tagged caspase-3 enzyme (Sigma-Aldrich®, Israel) was incubated with WPE, WSE, pith extract or bark extract and assayed in 96-well multiwall plate (50 ng/well), using the Caspase-3 Substrate II, Fluorogenic to determine the enzyme's fluorescent product. As a control a known irreversible pan-caspase inhibitor (CFI) was used. The assay was performed at 37° C. for 40 minutes, in a total volume of 100 μL.

### Viability Assay

HSOC viability assay—Determination of epidermal viability was carried out on separated epidermis tissues by using MTT assay (Thiazolyl Blue Tetrazolium Bromide, Sigma-Aldrich®; Cat. No.: M5655). The epidermis pieces were placed in 96-well plate and incubated at 37° C. for 1 hour, each well containing 120 μL 5 mg/mL MTT solution. The resulting precipitants (purple formazan crystals) were solubilized in 120 μL 2-Propanol for 15 min at room temperature. Then, the absorbance of the colored solution was measured using microplate reader at 560 nm (Multiskan EX, Thermo Scientific).

### Lipid Peroxidation End-Products

UVB induced lipid peroxidation in epidermal layer was determined using fluorescence assay quantifying malondialdehyde (MDA) levels (OxiSelect™ TBARS Assay Kit; Cell Biolabs, Inc.; Cat. No.: STA-330). The epidermis pieces were homogenized, and the sample was assayed directly for its thiobarbituric acid reactive substances (TBARS) level, according to the supplier's instructions. The fluorescent end-product was examined in a 96-well 96F Nunclon Delta Black Microwell SI (Thermo Scientific; Cat. No.: 137101) using a microplate reader Tecan Infinite M200 PRO.

### LPS-Induced Proinflammatory HSOCs Simulation

For HSOCs, a state of inflammation was mimicked ex-vivo by the addition of endotoxins (lipopolysaccharides) from E. coli (LPS, Santa Cruz Biotechnology, Inc.; Cat. No.: sc-3535) and Epithelial Growth Factor (EGF) in media (Life Technologies Ltd., Gibco®; Cat. No.: 41965-039) supplemented with 1% antibiotics Penicillin-Streptomycin Amphotericin B Solution (Biological Industries Israel Beit-HaEmek Ltd., Cat. No.: 03-033-1B); LPS containing solution was added systematically to the media. WPE or WSE were applied topically on HSOCs after obtaining the inflammatory state and co-incubated for 48 hours without reconstitution of the media. Two days after the topical applying of WPE or WSE the media were collected and analyzed as described hereinbelow.

### Quantification of Inflammatory Cytokines Following LPS-Induced Stimulation in HSOCs

The effect of WPE or WSE on the inflammation process was be determined by measuring the level of inflammatory cytokines, i.e., IL-6, IL-8 and TNFα, in the culture media of HSOCs that were previously incubated with the WPE or WSE. Specifically, collected DMEM samples for determining IL-6 and IL-8 were diluted 1:500 in fresh medium and transferred to the plate, while the collected DMEM sample for determining TNFα was transferred to the plate without being diluted. The amounts of specific cytokines in each sample was determined by sandwich enzyme-linked immunosorbent assay (ELISA) (Max™ Set Deluxe, BioLegend®; San Diego, Calif., U.S.) according to the manufacturer's protocol. The resulting product was quantitatively measured by optical density (OD) scanning at 450 nm with a Tecan Infinite M200 PRO reader.

### Determination of the Free-Radical Scavenging Activity in the 2,2-Diphenyl-1-Picrylhydrazyl (DPPH) Assay

The 2,2-diphenyl-1-picrylhydrazyl (DPPH) assay (Sigma-Aldrich®; Cat. No.: D9132) is used to monitor chemical reactions involving radicals. A working solution of 100 μM DPPH was prepared; 20 μL of WPE or WSE were added to 380 μL of DPPH solution in 24 wells plate; incubated in dark conditions for 30 minutes at room temperature to generate free-radicals; 100 μL of reaction solution was transferred to 96-well plate and the absorbance at 517 nm for each sample was measured on microplate reader Tecan Infinite M200 PRO. The scavenging effect of the sample solution to the DPPH radical solution was measured and converted to Trolox equivalent antioxidant capacity (TEAC), by which the results are presented.

### Electrospray Ionization Time of Flight Mass-Spectrometry (ESI-TOF-MS)

Mass spectrometry analysis was performed on samples using positive ESI-TOF-MS, at constant flow rate. Data interpretation was performed by the MassHunter Profinder software.

### Data Analysis

Data is expressed as mean±the standard error of the mean (SEM). Statistical analyses were performed using a 2-tailed Student's t-test. P-value of less than 0.05 (P<0.05) was considered a significant difference from the untreated control.

### Example 1

### Comparison of VOC in WPE and WSE

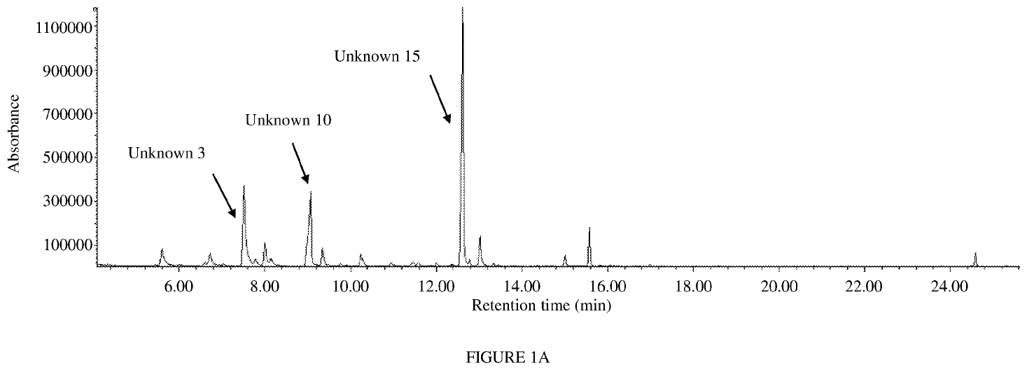

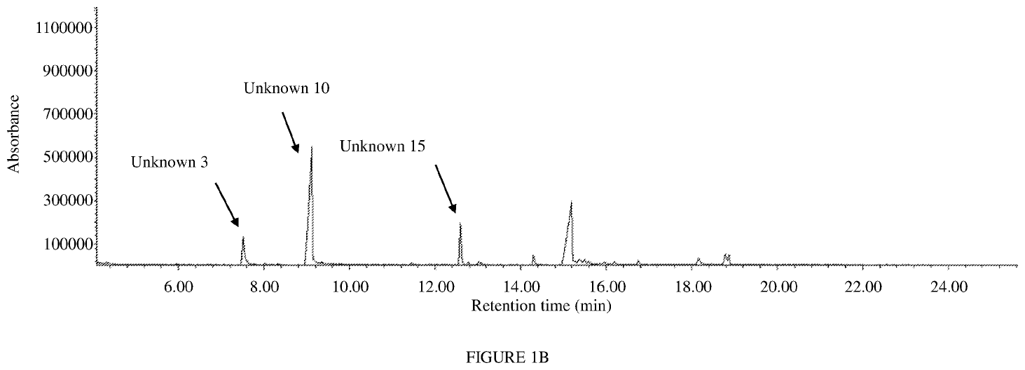

The inventors characterized VOC in WPE and WSE using gas chromatography. Certain peaks were highly prominent and shared between the WPE and WSE (FIGS. 1A-1B). Comparison of VOC in the WPE and WSE is summarized in hereinbelow (Table). WPE was found to comprise approximately 2.5-fold more VOC compared to WSE. Specifically, VOCs termed unknown 3 and unknown 15 were found to be more prominent in the WPE compared to WSE, while VOC termed unknown 10 was found in comparable amounts in both WPE and WSE (FIGS. 1A-1B).

Comparison of VOC in WPE and WSE (VOC of 0.5% w/w or less are
not presented)

| | WSE | WPE | WSE | WPE |
|---|---|---|---|---|
| Compound | % | % | μg/gr | μg/gr |
| a-Pinene | 4.8 | | 5.1 | |
| Sabinene | 0.7 | | 0.8 | |
| b-Pinene | 3.1 | | 3.3 | |
| unknown 1 | 1.7 | | 1.8 | |
| p-Cymene | 4.4 | 0.4 | 4.7 | 1.0 |
| unknown 2 | 2.1 | | 2.2 | |
| g-Terpinene | 3.8 | | 4.0 | |
| unknown 3 | 13.3 | 43.3 | 14.2 | 117.3 |
| unknown 4 | 3.0 | | 3.2 | |
| unknown 5 | 1.7 | | 1.8 | |
| unknown 6 | 0.9 | | 0.9 | |
| unknown 7 | 0.8 | | 0.8 | |
| unknown 8 | 0.6 | | 0.7 | |
| unknown 9 | 0.6 | | 0.6 | |
| unknown 10 | 41.2 | 7.8 | 44.0 | 21.3 |
| unknown 11 | 0.8 | 0.5 | 0.9 | 1.3 |
| unknown 12 | 5.1 | 0.6 | 5.4 | 1.6 |
| unknown 13 | | 1.5 | | 4.1 |
| unknown 14 | 1.8 | | 1.9 | |
| unknown 15 | | 33.1 | | 89.7 |
| unknown 16 | | 1.5 | | 4.1 |
| unknown 17 | | 1.0 | | 2.7 |
| Bornyl acetate | 4.6 | 0.5 | 5.0 | 1.4 |
| unknown 18 | | 0.7 | | 1.8 |
| unknown 19 | | 0.9 | | 2.4 |
| Piperonal | 0.3 | | 0.3 | |
| Pyrogallic acid | | 2.1 | | 5.6 |
| unknown 20 | | 2.6 | | 7.1 |
| unknown 21 | | 1.7 | | 4.6 |
| Benzophenone | 1.5 | | 1.6 | |
| TOTAL | 96.8 | 98.2 | 103.2 | 265.9 |

### Example 2

### Comparison of Antioxidant Activity and Total Phenol Content in WPE and WSE

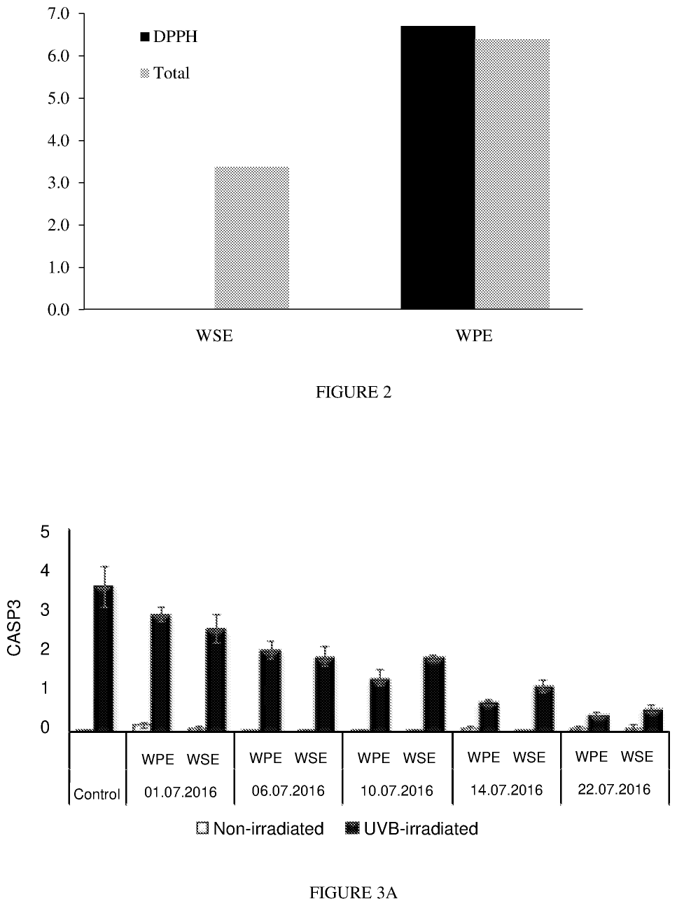

The inventors quantified the antioxidant activity and the total phenolic content in both WPE and WSE. Both WPE and WSE were found to comprise phenolic compounds, with WPE comprising approximately 2-fold more compared to WSE (FIG. 2). WPE had radical scavenging activity in the DPPH assay with an antioxidant capacity of 6.7 mg/Trolox equivalent per gram dry weight. Antioxidant capacity was not detected for WSE.

### Example 3

### WPE and WSE Attenuate UVB-Induced Apoptosis in HSOCs

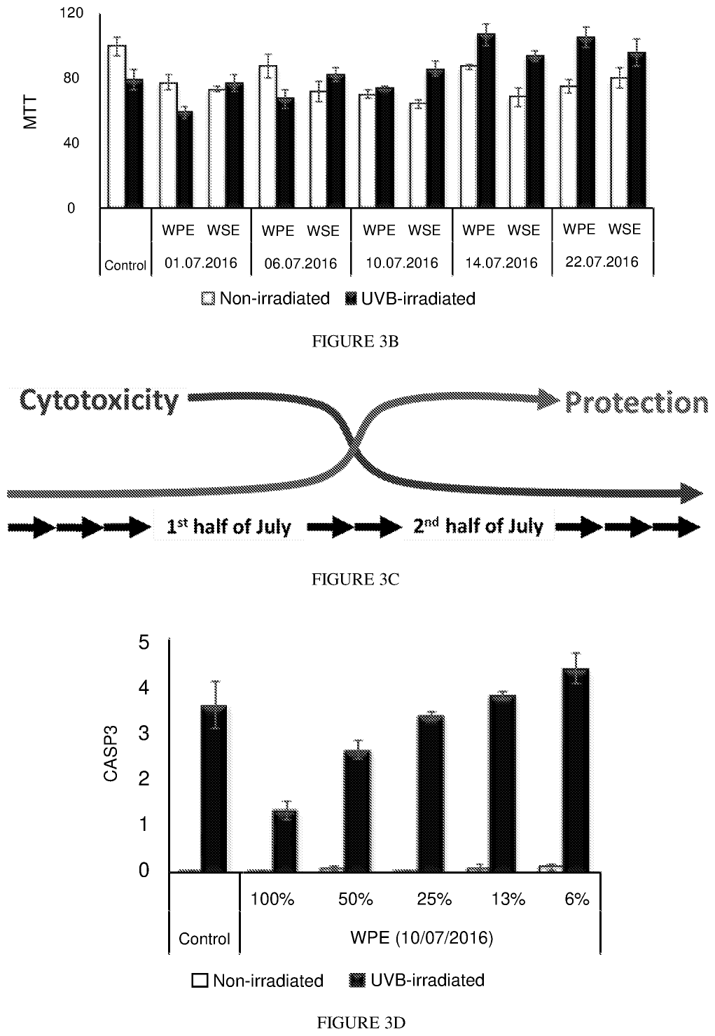

The inventors found that the chemical composition of C. gileadensis plant changes in a relatively short period of time, from July to August. Indeed, in the beginning of July, WPE and WSE possessed a photoprotective potential, but it was still far from its maximum (FIG. 3A). In addition, during this period, WPE and WSE were found to be highly toxic for HSOCs (FIG. 3B). But, by the middle of July, cardinal changes began to occur—the ability to prevent photodamage was increased, and at the same time the cytotoxicity of the substance decreased. Such a trend continued, and by the beginning of August the emulsions reached their maximum activity of protecting the HSOCs from harmful UV radiation. It is important to emphasize that during this period, the emulsion cytotoxicity, if not completely gone, has reached acceptable limits.

The inventors concluded that between the Pt and 2nd half of July, important processes occurred in the plant that allowed enhancing the photoprotective component and reducing toxicity (FIG. 3C). Furthermore, regarding WPE, its effect was demonstrated to be dose dependent (FIG. 3D).

The inventors then showed that the period July-August, is most critical for the final maturation of the plant and the achievement of the highest degree of biological activity. On this note and with respect to components of the WPE, the inventors showed that the main reservoir of the active substance, which is responsible for the photoprotective activity and cytotoxicity, is in the bark of the plant, and not in its pith (FIG. 3E).

### Example 4

### C. gileadensis Retains its Photoprotective Ability Under Multiple Irradiations

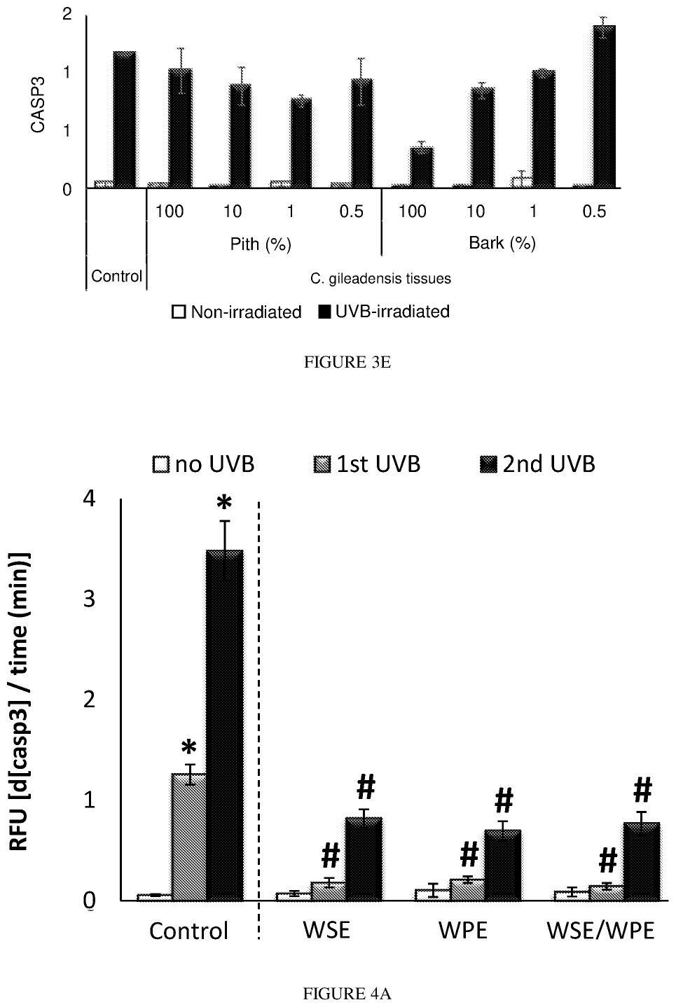

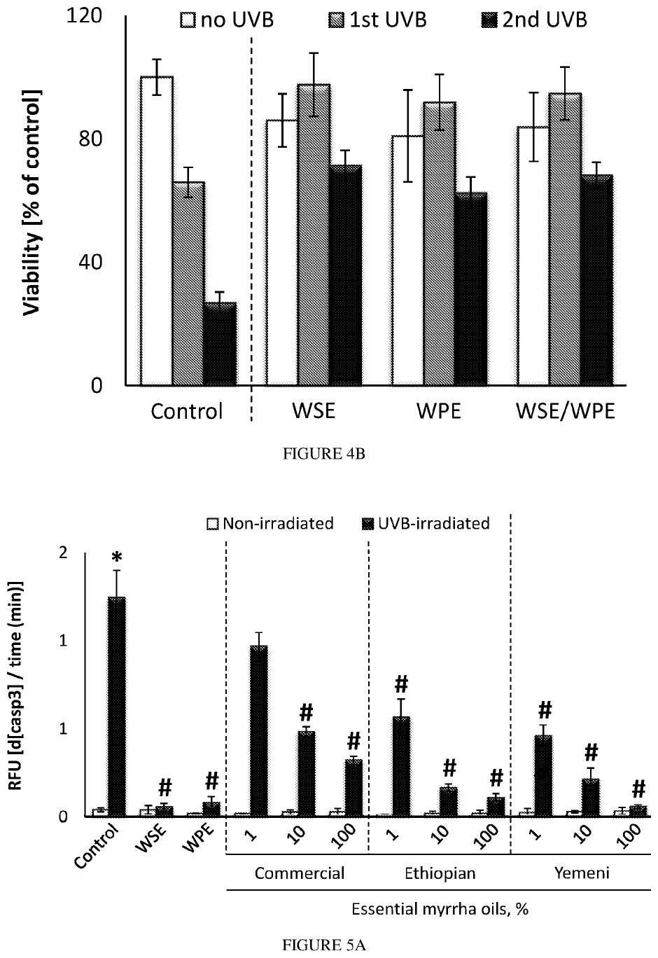

Continued testing of the properties of WPE and WSE of C. gileadensis showed impressive results in protecting HSOCs from harmful UV radiation (FIGS. 4A-4B). The HSOCs were pretreated with WSE, WPE and mixture of WSE/WPE), and irradiated after incubation. The next day the samples were subjected to repeated irradiation. The peculiarity of this experiment was that it consisted of two groups: (i) after the first irradiation (standard treatment); (ii) after double exposure. Evaluating the photoprotective activity of the extracts showed high efficiency of both WPE and WSE in prevention of photodamages incurred by repeated irradiation.

### Example 5

### C. gileadensis WPE and WSE Proved to be Advantageous Over Other Available Ethereal Myrrha Oils

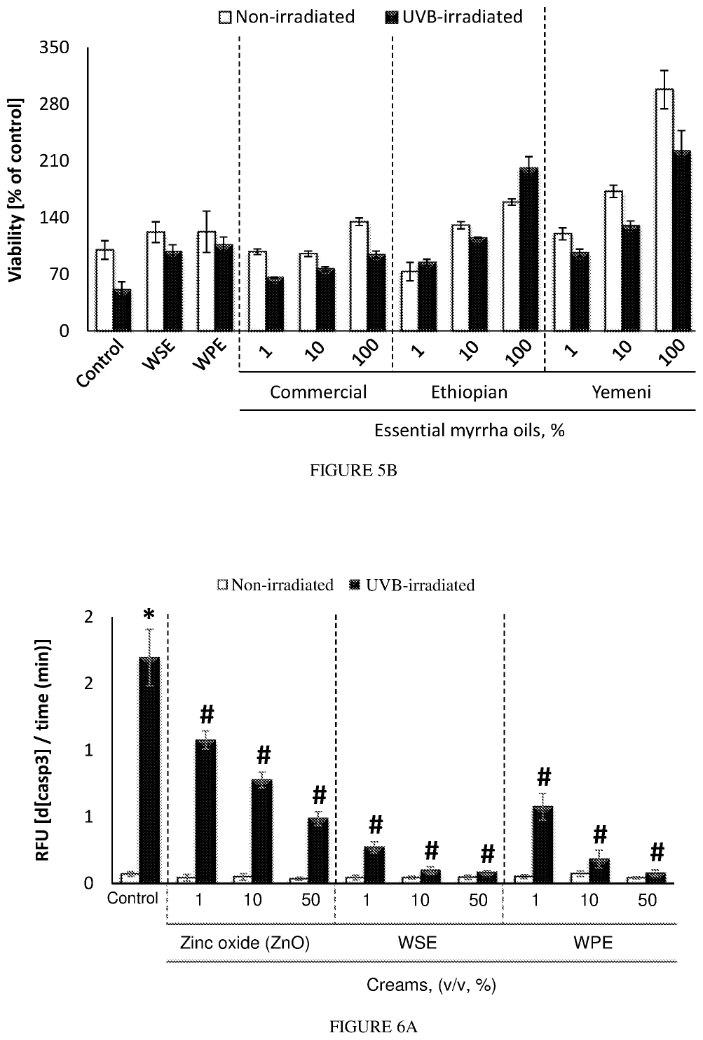

A comparative analysis of the disclosed WPE and WSE and available essential oils of myrrh, widely used in the cosmetic industry: commercial oil; oil produced in Ethiopia; and original oil from Yemen (FIGS. 5A-5B). The results prove that the emulsified extracts produced in the Ein-Gedi region (during August 2016) had undoubted advantages over the other extracts. For example, diluted commercial oil was less active and rapidly lost its photoprotective ability (FIGS. 5A-5B).

### Example 6

### C. gileadensis WPE and WSE Demonstrated their Advantage Over Commercial Sunscreens

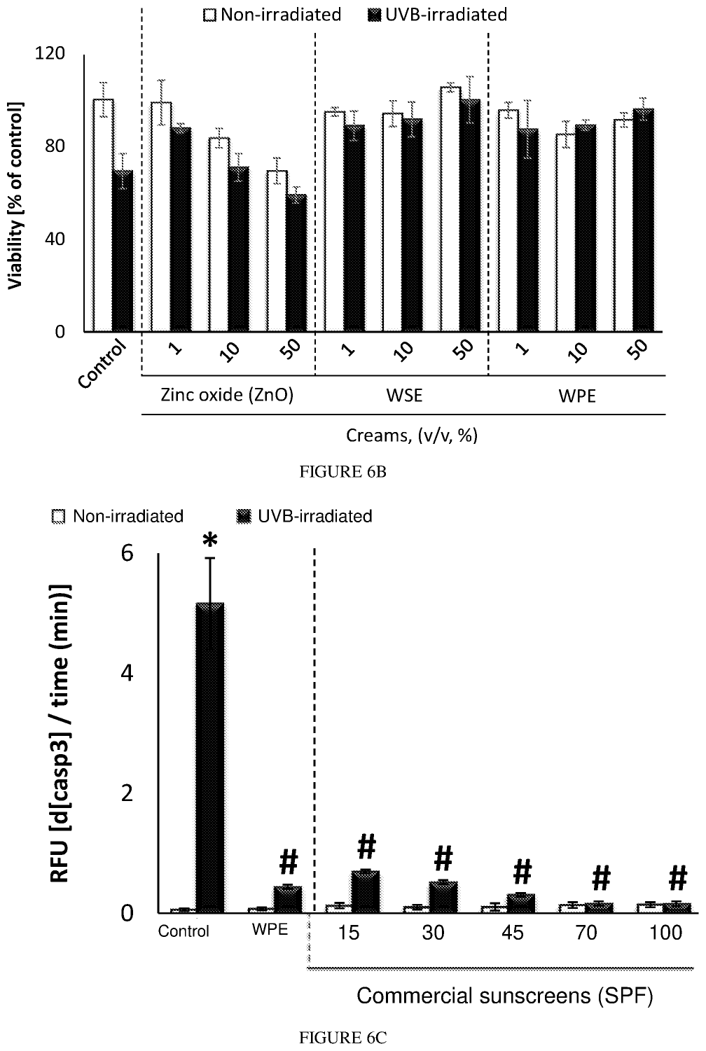

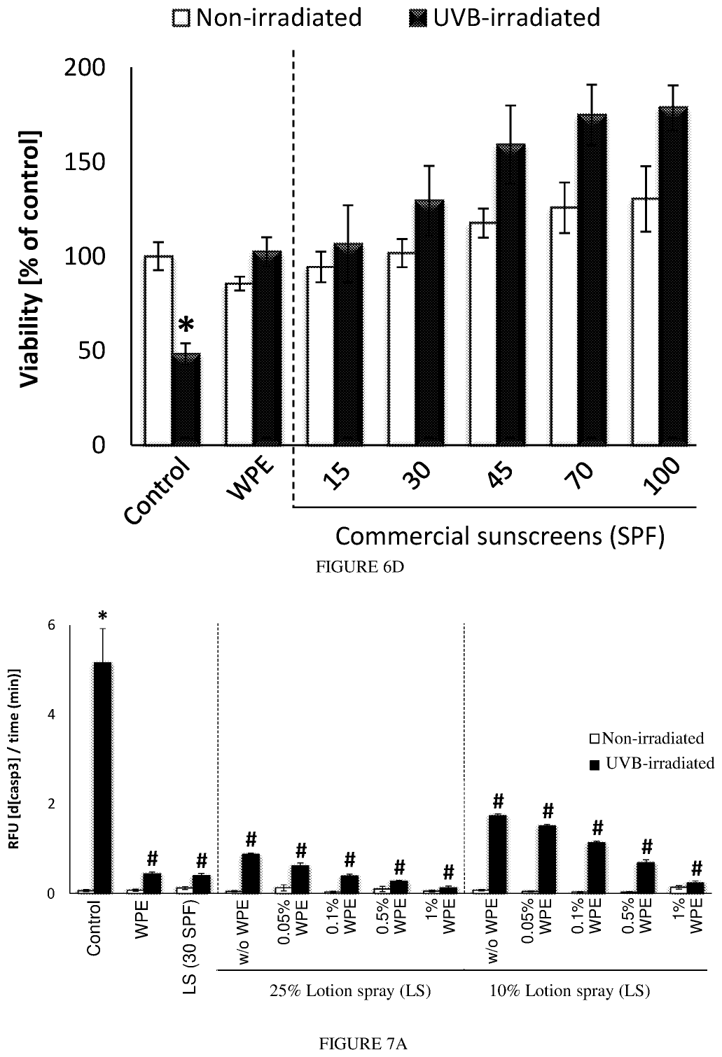

In addition, the inventors conducted a comparative analysis of creams containing zinc oxide (ZnO), widely used cosmetic procedures and sunscreens as UV absorbers, or WPE and WSE prepared in August (FIG. 6A-6B). It was noticeable that zinc oxide had less photoprotective activity and greater cytotoxicity than organic cream comprising WPE or WSE. A similar result was observed in the following experiment testing the photoprotection and toxicity of popular mineral creams with various SPFs: from 15 to 100 (FIGS. 6C-6D). It was confirmed that commercial mineral creams did have powerful photoprotective ability, and at the same time had a cytotoxic effect expressed by the increase in mitochondrial activity. On the contrary, WPE (of an August harvest) had the maximum photoprotective activity simultaneously with reduced toxicity.

### Example 7

### C. gileadensis WPE and WSE have Synergistic Photoprotecting Effect when Added to Sunscreen Lotion

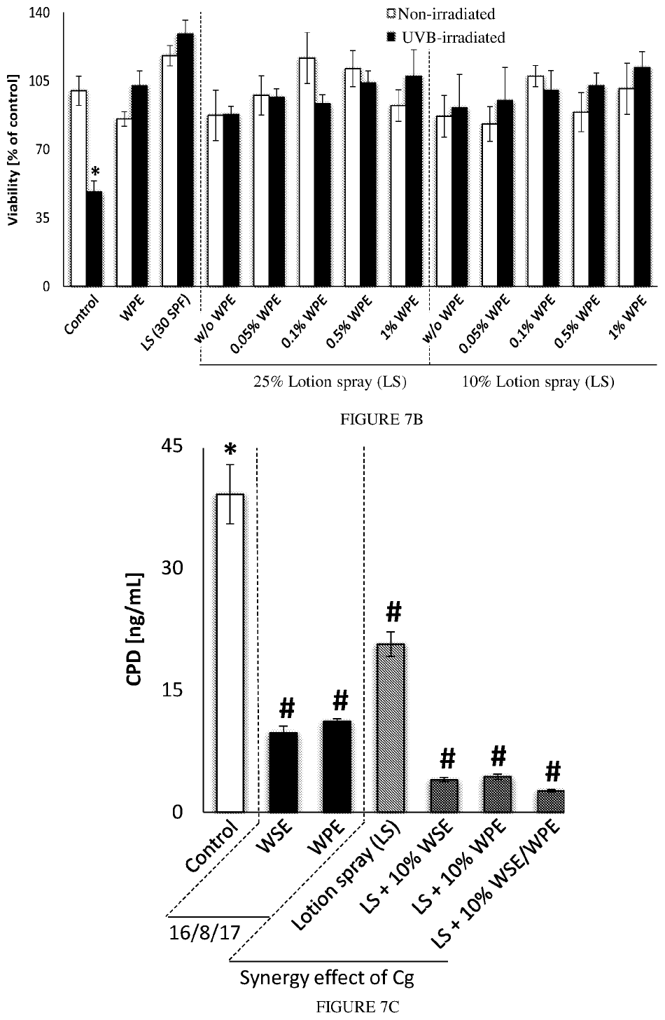

The inventors then wanted to test if there is a synergistic effect by combining C. gileadensis WPE with synthetic sunscreens. When WPE was added to a sunscreen lotion a combination of the minimum concentrations of both ingredients resulted in a maximum protective effect with a balanced viability (FIGS. 7A-7B). In a following experiment, the possibility of a synergistic interaction in preventing the formation of pyrimidine dimers (CPD) was also tested. The inventors clearly demonstrated that the use of various mixtures combining sunscreen lotion and WPE, WSE, or both, enhanced the protective effect under the action of ultraviolet rays and prevented the appearance of a mutagenic DNA defect, as compared to the effect induced by each one of them when used separately (FIG. 7C).

### Example 8

### C. gileadensis WPE and WSE have a Dual Anti-Inflammatory Effect

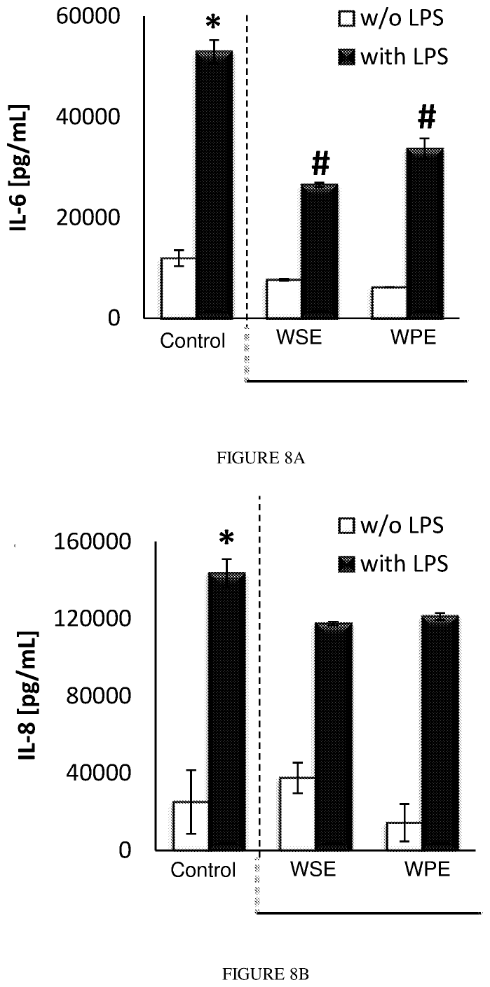

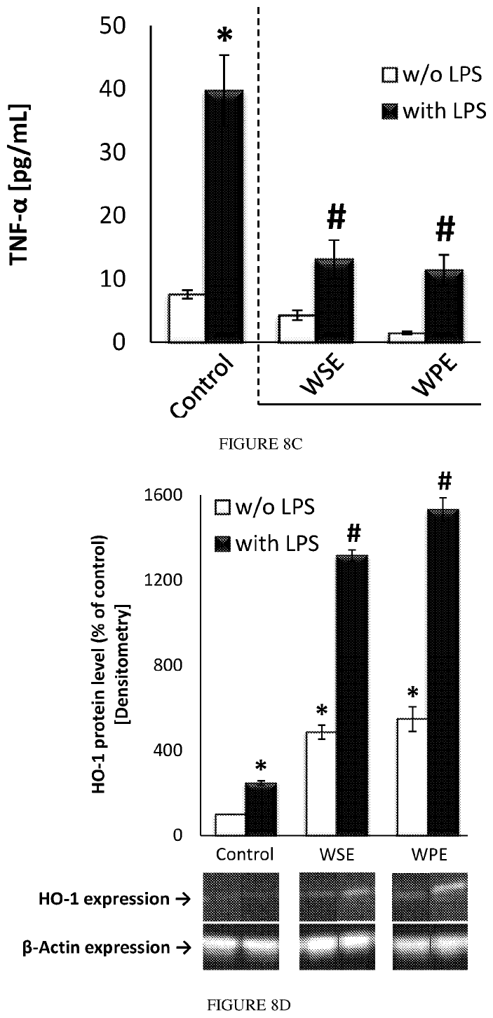

Expression of pro-inflammatory cytokines after induction of inflammation in the HSOCs by LPS and EGF and exposure to WPE and WSE was examined (FIGS. 8A-C). WPE and WSE were able to cause only a slight decrease in the production of IL-6 (FIG. 8A); in turn, IL-8 expression was practically non-affected (FIG. 8B). On the contrary, TNFα synthesis was significantly decreased (FIG. 8C). The inventors also showed that WPE and WSE induced expression activation of the enzyme hemoxygenase-1 (HO-1), known to have cryoprotective properties (FIG. 8D).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising a water-based extract of a Commiphora plant, and optionally a cosmetically acceptable carrier.

2. The composition of claim 1, obtained by water-based extraction of said Commiphora plant.

3. The composition of claim 1, wherein said Commiphora plant comprises:
a. leaves: 10-30% (w/w) by dry weight;
b. fruits: 5-15% (w/w) by dry weight;
c. branches: 20-60% (w/w) by dry weight;
or any combination thereof.

4. The composition of claim 1, comprising at least 10% (w/w) sap by dry weight.

5. The composition of claim 1, comprising 0.2 to 2% (w/w) of a phenolic compound.

6. The composition of claim 1, comprising 0.01 to 0.05% (w/w) of a volatile organic compound (VOC).

7. The composition of claim 1, wherein said Commiphora is Commiphora gileadensis.

8. The composition of claim 1, comprising a fraction of said water-based extract.

9. The composition of claim 1, further comprising an anti-oxidant, a chelator, a cleansing agent, a skin protectant, a sunscreen, a skin lightening agent, an anti-wrinkling agent, an anti-inflammatory agent, an anti-aging agent, or any combination thereof.

10. (canceled)

11. A method for preventing or treating an ultra-violate (UV) radiation damage to a subject's skin, comprising topically applying to said subject's skin a composition comprising an effective amount of a water-based extract of Commiphora plant, and optionally wherein said composition further comprises a cosmetically acceptable carrier.

12. The method of claim 11, wherein said Commiphora plant comprises:
a. leaves: 10-30% (w/w) by dry weight;
b. fruits: 5-15% (w/w) by dry weight;
c. branches: 20-60% (w/w) by dry weight;
or any combination thereof.

13. The method of claim 11, wherein said composition comprises at least 10% (w/w) sap by dry weight.

14. The method of claim 11, wherein said composition comprises 0.2 to 2% (w/w) a phenolic compound.

15. The method of claim 11, wherein said composition Amended 0.01 to 0.05% (w/w) a VOC.

16. The method of claim 11, wherein said Commiphora is Commiphora gileadensis.

17. The method of claim 11, wherein said composition comprises a fraction of said water-based extract.

18. The method of claim 11, wherein said composition is topically applied to said subject's skin before, during, or after exposure to UV radiation, and optionally wherein said UV radiation is UVB radiation.

19. (canceled)

20. The method of claim 11, wherein said subject is at risk of sunlight exposure.

21. (canceled)

22. The method of claim 11, wherein said composition is in the form of a cream, a lotion, an ointment, or a spray.

23. The method of claim 11, wherein said composition further comprises an anti-oxidant, a chelator, a cleansing agent, a skin protectant, a sunscreen, a skin lightening agent, an anti-wrinkling agent, an anti-inflammatory agent, an anti-aging agent, or any combination thereof.
